(19) United States
(12) Patent Application Publication
(10) Pub. No.: US 2002/0019006 A1
(43) Pub. Date: Feb. 14, 2002

(54) Proteomic interaction arrays

(75) Inventors: Olive Yi-lu Yuan, Arlington, VA (US); Dario B. Valenzuela, Boxborough, MA (US); Peter Rapiejko, Upton, MA (US); Heidi M. Hoffmann, Lunenburg, MA (US); kara Ann Higgins, Billerica, MA (US)

Correspondence Address: Doreen M. Hogle, HAMILTON, BROOK, SMITH & REYNOLDS, P.C., Two Militia Drive, Lexington, MA, 02421-4799, US

(73) Assignee: AlphaGene,Inc., Woburn, MA

(21) Appl. No.: 09/922,441

(22) Filed: Aug. 3, 2001

Related U.S. Application Data
(63) Continuation of application No. PCT/US00/02974, filed on Feb. 4, 2000.
(60) Provisional application No. 60/118,901, filed on Feb. 5, 1999.

(51) Int. Cl. C12Q 1/68

(52) U.S. Cl. 435/6

(57) ABSTRACT

A method is provided for the rapid identification of protein-protein interaction networks within a cell, tissue, or whole genome. The introduction of a multi-bait approach is a distinguishing feature of the technology. In this method a pair of two-hybrid cDNA libraries, each one carrying the complement of genes from the tissue under study, are combined for an interaction screen. A large number of yeast colonies, each identifying a protein interaction pair, are picked and distributed in single wells, providing an arrayed archive of protein-protein interactions. The archive also serves as a source of plasmids to construct arrayed replicas containing DNA of the interacting plasmid pairs. Hybridization of a given cDNA to the arrayed replicas identifies the corresponding interacting clones. Protein interaction networks are constructed by iteration of the hybridization with newly identified interacting clones.

### RELATED APPLICATIONS

This application is a continuation of International Application No.: PCT/US00/02974, filed on Feb. 4, 2000, which claims the benefit of U.S. Provisional Application No. 60/118,901, filed Feb. 5, 1999, the teachings of which are incorporated herein by reference in their entirety.

### BACKGROUND OF THE INVENTION

The identification of new pathways involving protein-protein interaction in disease states has a high commercial value because of their potential to identify therapeutic targets. A variety of procedures have been developed to identify interactions between proteins. Three common biochemical methods to screen for interacting proteins are, co-immunuprecipitation, affinity chromatography, and expression library screening. Coimmunprecipitation is one of the most common biochemical methods to search for interacting proteins. For example, the well-known interaction between retinoblastoma protein (p110^{RB}) and adenoviral protein E1A was obtained using this approach (Whyte et al., Nature 334:124-129 (1988)). Affinity chromatography typically involves a bait protein linked to beads. Proteins that interact with a bait protein bind the bait and are eluted after washing the column. A typical application is to use glutathione-S-transferase protein (GST) fused to a polypeptide of interest as the bait protein. Expression library screening involves screening the library using a labeled bait protein as a probe. Expression library screening has been successful in identifying genes encoding proteins that interact with calmodulin, jun, myc, the EGF receptor and the retinoblastoma protein. Although these methods have generated many significant results, they are all in vitro methods and are generally difficult to modify for high throughput. Furthermore, once the interacting proteins are identified, the cloning of its corresponding gene could be very challenging and not easily engineered for high throughput analysis on discovery.

An in vivo genetic approach to detect protein-protein interactions is the yeast two-hybrid system. The two-hybrid system is typically applied to detect the interaction between two proteins or to isolate interacting proteins from a library using a specific bait.

The two-hybrid system permits an in vivo identification of the interacting proteins. Hence, the conformation of the target protein in yeast cells is closer to the native form than most of the in vitro conditions that are available, and reasonably it is therefore more likely to yield physiologically significant proteins. It is likely to be more sensitive for detection of protein-protein interaction than many other methods, such as probing an expression library with a labeled protein or co-immunoprecipitation, based on the parallel comparisons (Li et al., FASEB J. 7:957-963 (1993)). This sensitivity allows the isolation of weaker or transiently interacting proteins. Numerous protein interactions have been successfully detected by using the two-hybrid system, including cell cycle factors, signal transduction factors, proteins involved in apoptosis and DNA repair.

The use of the two-hybrid approach to determine protein-protein interaction rapidly and on a large scale has certain obstacles. Modification for high throughput analysis of protein-protein interaction or high throughput identification of novel interacting proteins requires a tremendous amount of labor intensive subcloning of gene sequences of interest and sequencing of newly discovered candidate genes from libraries, making high throughput two-hybrid approaches time consuming and expensive. Traditionally, two-hybrid screens are performed using a single bait protein, screening nucleic acids that encode potentially interacting proteins, retrieving the clones encoding the interacting proteins, sequencing these clones, and storing the sequence information in silico. The sequencing cost is extremely high when a genome-wide two-hybrid screen performed.

Furthermore, it has been estimated that the human genome encodes 100,000 proteins with potentially 50 billion different protein-protein interactions. However, not all of these interactions are expected to be involved in pathways that are either involved in disease or development or are suitable drug targets. Thus, analysis of all two-hybrid interacting pairs involves considerable time, effort and expense for interactions that may have little or no commercial or research value.

Another limitation of the traditional two-hybrid approach has been the inability to reconstitute interactions mediated by several components or interactions that are dependent on specific post-translational modifications. Several assays have been described to overcome this barrier, including co-expression of a protein tyrosine kinase as a modifying enzyme to assess the interactions between phosphoproteins. However, these studies typically focus on a single bait protein and the interactions in the presence of the third protein, either as a modifier or stabilizer.

Further, limitations of the two-hybrid approach are the presence of false positives and false negatives. Although improvements have been implemented to reduce the number of false positives, the problem still exists. From the bacteriophage T7 protein linkage mapping project, it was found that the large majority of false positives appear to be due to transcriptional activation from the DNA binding domain clones in the absence of protein-protein interaction. That is to say, the DNA-binding domain hybrid (BD-X) activates the reporter gene by itself. In addition, false positives can result from the non-specific interaction via short stretches of residues.

### SUMMARY OF THE INVENTION

The present invention is drawn to a method of selecting nucleic acids encoding polypeptides, wherein the polypeptides are capable of interacting, in vivo (e.g., in a living cell) with a polypeptide of interest. The method comprises providing or generating an array of plasmid partners and probing the array with nucleic acid encoding a polypeptide of interest. Polynucleotides encoding a polypeptide that interacts with the polypeptide of interest are identified by contacting the array with polynucleotide probe encoding all or a portion of said polypeptide of interest under conditions where the probe detectably hybridizes to complementary sequence, if present, within any of the plasmids of the array. Plasmid partner or partners of the hybridized plasmid are identified and optionally isolated, wherein said partner or partners encode a polypeptide capable of interacting, under physiological conditions, with the polypeptide of interest.

The plasmid partners comprise two or more plasmids wherein each plasmid comprises a polynucleotide sequence encoding a polypeptide fused or linked to nucleic acid sequence encoding a DNA binding protein domain or a transcriptional activation domain. The plasmid partners are selected to be in the array by their ability to, in concert, produce a detectable biochemical readout, e.g., transcription of one or more marker genes in a host cell.

The present invention is drawn to a method of isolating polynucleic acids encoding at least one polypeptide capable of interacting, in vivo with a polypeptide of interest comprising:

a) contacting at least one array of plasmids with a probe, wherein:

i) said probe encodes the polypeptide of interest or fragment thereof, wherein said probe hybridizes to complementary sequence, if present, within any of the plasmids, and wherein:

ii) said array comprises two or more plasmid partners, wherein a first plasmid partner comprises a first library fused to a first nucleic acid sequence encoding a first half of a selection pair and a second plasmid partner comprises the same or a second library fused to a second nucleic acid sequence encoding a second half of a selection pair and wherein the plasmid partners are selected to be in the array by their ability to, in concert, activate the selection pair in a host cell, and

b) identifying the partner or partners to said hybridized plasmid, wherein said partner or partners encode a polypeptide capable of interacting, in vivo with the polypeptide of interest.

The present invention is drawn to a method of generating an array of plasmids comprising:

a) conducting a two-hybrid screen, wherein bait plasmids comprise a cDNA library and a prey plasmids comprise the same or a second cDNA library;

b) selecting positive two-hybrid clones and

c) immobilizing the bait and prey plasmids from said positive clones on a solid support at known locations, thereby generating an array of plasmids.

In another embodiment of the present invention, a method is provided for selecting polypeptides capable of interacting in vivo with a polypeptide of interest, wherein the interaction is dependent upon post-translational modification. The method comprises providing or generating arrayed sets of plasmids comprising three or more plasmid partners, wherein a first plasmid partner comprises a library of sequences encoding polypeptides fused or linked to nucleic acid sequence encoding a DNA binding domain and wherein a second plasmid partner comprising the same or a second library fused or linked to nucleic acid encoding a transcriptional activation domain and wherein a third plasmid partner comprises at least one post-translational modifying enzyme. The expression of said enzyme is optionally under the control of an inducible transcription system. The first and second plasmid partners are selected by their ability, in concert, and in the presence of the expressed post-translational modifying enzyme, activate transcription of one or more marker genes in a host cell. Polypeptides that interact with the polypeptide of interest are selected by contacting nucleic acid encoding the polypeptide of interest to the array, under conditions where the nucleic acid detectably hybridizes to complementary sequence, if present, within any of plasmids. Plasmid partner or partners of the hybridized plasmid are identified and optionally isolated, wherein said partner or partners encode a polypeptide capable of interacting, in vivo and in a post-translational modification dependent manner, with the polypeptide of interest.

In another embodiment of the present invention, a method is provided for selecting polypeptides capable of interacting under physiological conditions, with a polypeptide of interest, wherein the interaction is inhibited by post-translational modification. The method comprises providing or generating arrayed sets of plasmids comprising three or more plasmid partners, wherein a first and second plasmid partner as described above and a third plasmid partner comprising at least one post-translational modifying enzyme. The expression of said enzyme is optionally under the control of an inducible transcription system. The first and second plasmid partners are selected by their ability to, in concert and in the absence of the post-translational modifying enzyme, activate transcription of one or more marker genes in a host cell. The array is probed with a polynucleotide encoding polypeptide of interest as described above.

The present invention provides a powerful tool to generate complete linkage map of proteins encoded by the cDNA library used to create the plasmid partner array. This information can be stored in the form of a DNA chip, avoiding the high cost of sequencing all clones. The method of the present invention is particularly suitable for high throughput operation. The archive of arrays of the present invention allows identification and retrieval of the X, Y, or both sequences using standard hybridization techniques. A sequence of interest is used to probe the arrays of the linkage map by hybridization. A positive signal reveals both the sequence homologous to the probe as well as the partner plasmid. By using the linkage map of the present invention, one can probe the map with a sequence encoding a protein of interest, which hybridizes selectively to complementary sequences, when present, in the X or Y inserts and find a corresponding plasmid that encodes a protein that interacts with the protein of interest. For example, if the sequence encoding the protein of interest hybridizes to X₁, then the location of Y₁ in the array will be provided by the linkage map. The X₁ or Y1, sequence provided by the map can be used to probe for other interacting proteins.

In the method of the present invention, any pair of DNA-binding domains and activation domains can be used. Furthermore, any site-specific transcription factor that has separable DNA binding domain and activation domain can be used. Other methods to identify protein-protein interactions in the two hybrid-derived system include other reconstitutive methods whereby two domains that together produce a biochemical readout can be physically separated such that the reassociation of the separated domains via protein X and Y interaction reconstitutes the biochemical readout; for example, as reviewed by Mendelsohn and Brent (the teachings of which are incorporated by reference herein in their entirety). The membrane binding and catalytic domains of guanine exchange factor can be used (Mendelsohn and Brent, Science 284:1948-1950 (1999)). These separated domains are referred to herein as a first and second half of a selection pair, respectively.

The present invention is advantageous over other biochemical methods for a number of reasons. The present invention uses arrays to store all protein-protein interaction information identified using two-hybrid screening without performing a large amount of subcloning or sequencing. Instead, the positively interacting pairs are identified using probes selected by the user to hybridize the array and identify all locations of the array that contain DNA encoding the protein of interest and partner plasmids in the same or linked array that encode proteins that interact with said protein of interest. Therefore, the present invention significantly reduces the cost of mapping protein interactions on a cellular, tissue or genome wide scale. Moreover, the arrays of the present invention provide an archive in which a user can obtain the interaction partners rapidly by DNA hybridization; a much faster and simpler technique than yeast two-hybrid screen. A database of interacting partners can be produced by storing the identity of partner pairs identified by hybridization in computer table. The modification of using different selection markers on bait and prey vectors in the two-hybrid system simplifies the plasmid DNA recovery process and speeds up the entire two-hybrid screening procedure. In addition, the process of generating the array, probing the array and identifying plasmids encoding interacting pairs of proteins can be automated.

### BRIEF DESCRIPTION OF THE DRAWINGS

FIG. 1 is a schematic diagram of the generation of Proteomic Interaction Arrays.

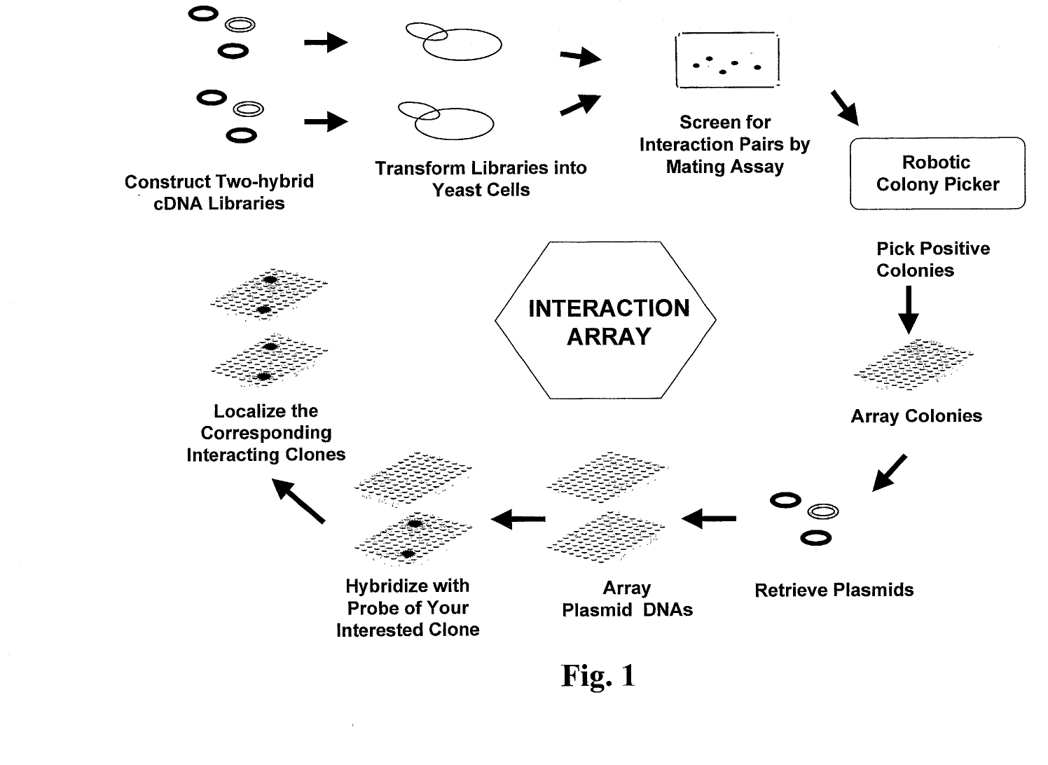

FIG. 2 is a diagram of two plasmids for use in the two-hybrid screen of the present invention.

FIG. 3 is a schematic diagram of the generation of Proteomic Interaction Arrays using three protein, two-hybrid (3PTH).

FIG. 4 shows the hybridization of vector sequences to the test array of Example 1.

FIG. 5 shows the hybridization of an Snk probe to the test array of Example 1.

FIG. 6 shows the hybridization of an B 11 probe to the test array of Example 1.

### DETAILED DESCRIPTION OF THE INVENTION

The human genome sequencing project has had a revolutionary effect on biological research. The decoding of all genes brings the blueprint of life to scientists, while the function of genes still remains a mystery. Since virtually all cellular processes are controlled by proteins, including disease processes and developmental processes, knowledge of how proteins interact is necessary to understand these processes.

The present invention relates to a novel high throughput approach to two-hybrid analysis. The yeast two-hybrid system is a genetic approach which allows one to detect protein-protein interaction in vivo through the reconstitution of the activity of a transcriptional activator, such as GAL4, in yeast Saccharomyces cerevisiae. The key of the two-hybrid system is the finding that site-specific transcription factors are often modular, comprised of separable DNA-binding domains (BDs) that bind to a specific promoter sequence, and activation domains (ADs) that direct the RNA polymerase II complex to transcribe the gene downstream of the DNA binding site. This phenomenon is exploited by fusing separate binding and activation domains to a pair of interacting proteins, X and Y, to create two hybrid proteins, BD-X and AD-Y. If the X and Y proteins interact, co-expression of two hybrids in a yeast cell leads to expression of a reporter gene containing the cognate BD-binding site. This approach can be also used to isolate cDNAs encoding partners for a protein of interest from an AD-Y library.

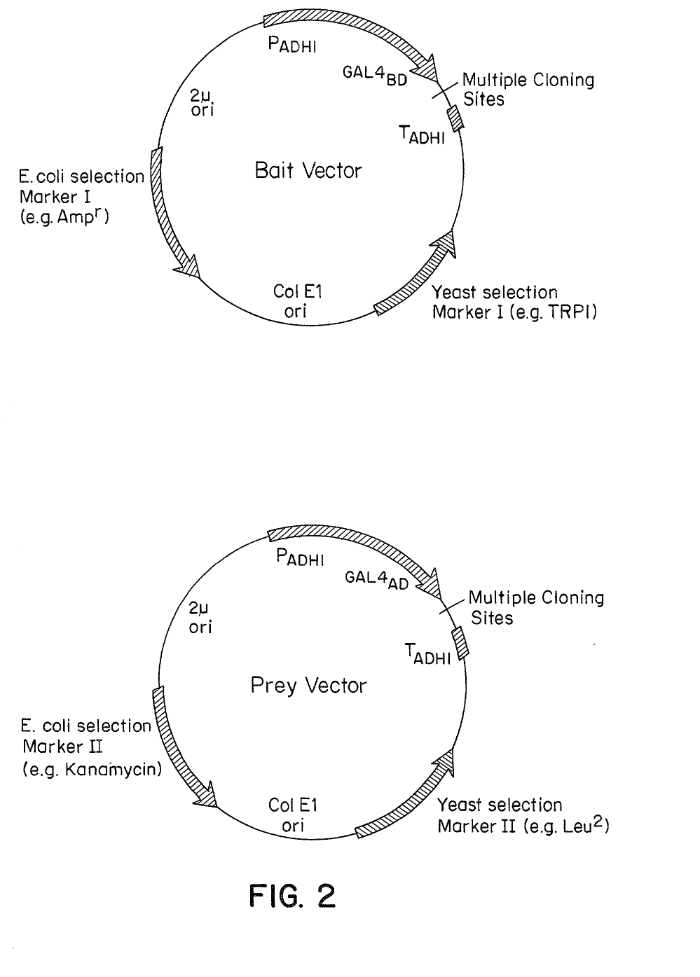

The present invention is drawn to a genome-wide scale two-hybrid screen. The method of the present invention involves a rapid plasmid retrieval system to recover plasmids from two-hybrid positive cells, an array system to store the interaction information using suitable substrates such as filter membranes or microarrays (eg. on glass slides), and a hybridization method to identity nucleic acid encoding proteins that interact with a polypeptide of interest. The present invention also relates to cDNA libraries constructed in suitable two-hybrid plasmids, such as the two plasmids shown in FIG. 2.

The present invention is drawn to a method to quickly identify DNA encoding proteins which interact with a protein of interest, by probing the arrays of the present invention with nucleic acid encoding the protein of interest. The method involves construction of an array of protein-protein interactions represented by plasmid pairs, wherein the plasmid pairs have been selected from a genome-wide scale two-hybrid screen. The collection of plasmid pairs is referred to herein as an “interaction library” or array. In this embodiment, the first and second plasmid pairs are generated using the cDNA library from a cell line or tissue of interest. In another embodiment, the array represents the entire complement of protein-protein interactions of an organism. The present invention can also be applied to other screens, such as yeast three-hybrid, one-hybrid, and mammalian two-hybrid screen.

In the method of the present invention, positive yeast hybrids from the two-hybrid assay are selected and distributed in an array, such as a two dimensional array. The clones are also stored for future access. Each yeast hybrid is a clone containing at least two plasmids. To identify nucleic acid encoding polypeptides that interact with a polypeptide of interest, the plasmids from the yeast clone array are transferred to a solid support for hybridization screening. The plasmid array is probed with a nucleic acid selected by a user. In one embodiment, the nucleic acid encodes a protein of interest. The array is contacted with the probe under suitable hybridization conditions. The wells or array locations containing nucleic acid homologous with specific probe are identified by hybridization with the probe. Vectors or plasmids encoding interacting partner(s) of the protein of interest are also identified. After identification of the vectors encoding interacting partners, the process can be repeated with the newly identified polynucleic acid molecules to reveal polynucleic acids encoding proteins that interact with the previously identified interacting partners. Thus, the method and arrays of the present invention provide networks of interaction pathways within a cell.

The skilled artisan will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target combination is often found by the well known technique of fixing several of the aforementioned stringency factors and then determining the effect of varying a single stringency factor. Optimal stringency for hybridizing the user defined probe to the array may be experimentally determined by examining variations of each stringency factor until the desired degree of discrimination between specific and non-specific sequences has been achieved. The level of stringency will increase or decrease depending on whether the target and variable regions are complementary or substantially complementary.

A general description of stringent hybridization conditions is provided in Ausubel, F. M., et al., Current Protocols in Molecular Biology, Greene Publishing Assoc. and Wiley-Interscience 1989, the teachings of which are incorporated herein by reference. The influence of factors such as probe length, base composition, percent mismatch between the hybridizing sequences, temperature and ionic strength on the stability of nucleic acid hybrids is well known in the art. Thus, stringency conditions sufficient to allow the user defined probes to hybridize with specificity to a homologous nucleic acid sequence, if present, in the array can be determined empirically. The probe need not hybridize to the nucleic acid sequence of interest with exact complementarity, so long as the target nucleic acid sequence of interest is identical or nearly identical to the probe, e.g., a homolgue or variant.

Conditions for stringency are also described in: Secreted Proteins and Polynucleotides Encoding Them, (Jacobs et al, WO 98/40404), the teachings of which are incorporated herein by reference. In particular, examples of highly stringent, stringent, reduced and least stringent conditions are provided in WO 98/40404 in the Table on page 36. In one embodiment of the present invention, highly stringent conditions are those that are at least as stringent as, for example, 1× SSC at 65° C., or 1× SSC and 50% formamide at 42° C. Moderate stringency conditions are those that are at least as stringent as 4× SSC at 65° C., or 4× SSC and 50% formamide at 42° C. Reduced stringency conditions are those that are at least as stringent as 4× SSC at 50° C., or 6× SSC and 50% formamide at 40° C.

The present invention expedites the process of discovering novel interaction pathways while minimizing the need for subcloning or sequencing polynucleotides that do not encode proteins that interact with a polypeptide of interest.

Also provided by the present invention are two-hybrid plasmids, one containing the DNA-binding domain (first plasmid) and the other containing the activation domain (second plasmid). These two plasmids use two different E. coli selection markers. Useful selection markers for the present invention include for example, genes encoding ampicillin, kanamycin, chloramphenicol, tetracycline, Zeocine and trimethoprim resistance. In one embodiment, three selection markers can be used. For example, a unique selectable marker can be present on each of the plasmids, such as ampicillin on plasmid one and kanamycin on plasmid two, and a common selectable marker present on both plasmids. In another embodiment, one marker can be used a common marker present on both plasmids. The markers allow the isolation of the plasmids using techniques well known in the art. For example, the plasmids isolated from the two hybrid clone can be transformed into E. coli which are grown in the presence of the appropriate antibiotic. Plasmids are purified from the selected E. coli using standard techniques in the art. A common marker would allow the isolation of both plasmids in a single step, saving time and expense involved in separate isolations.

In the method of the present invention, a protein-protein linkage map is generated and stored as an array, without the need for subcloning or sequencing the X or Y inserts. The present invention also provides a proteomic array of interactive protein pairs, and nucleic acid encoding the pairs and is also referred to herein as an archive. The interactive protein pair information can be stored as a database comprising at least one of said arrays, coupled with information describing the linkage between the members of said array. For example, each selected two-hybrid clone harbors a bait plasmid and a prey plasmid. These plasmids can be stored together in the same location of the two dimensional array, or bait plasmids from all two-hybrid clones can be stored in one array or set of arrays while the prey plasmids are stored in a separate array or set of arrays. The bait and prey arrays will be linked by information such that identifying a given bait or prey plasmid reveals the location of the corresponding prey or bait plasmid, respectively.

The arrays of the present invention can be of any suitable size on any suitable substrate. In one embodiment of the present invention, the arrays are two dimensional. In another embodiment, the substrate is a plastic tray or plate comprising wells, such as a 96 well or 384 well plate. Said plates are well known in the art. In another embodiment, the array can be a series of spots on a substrate, such as nylon membrane, glass slide, or photolithographic biochip. The amount of DNA in a given spot can be as little as nanogram quantities, however, less can be used depending on the sensitivity of the detection system. The number of spots in an array can be very large depending on the resolution of the spotting or printing apparatus used.

The two-hybrid systems and proteomic arrays of the present invention can be combined a microarray system. As a result, differentially expressed genes can be identified in normal or disease states, or at particular stages of development without having to examine 50 billion potential interactions. Methods of making microarrays are well known in the art and methods of identifying differentially expressed genes is described in U.S. Ser. No. 09/350,609, the teachings of which are incorporated herein by reference in their entirety.

In another embodiment of the present invention, a method is provided for selecting polypeptides capable of interacting in vivo with a polypeptide of interest, wherein the interaction is dependent upon post-translational modification (3 protein, two-hybrid). The method comprises providing or generating arrays comprising a first plasmid partner comprising a library linked to nucleic acid sequence encoding a DNA binding domain and a second plasmid partner comprising the same or a second library linked to nucleic acid encoding a transcriptional activation domain, wherein the first and second plasmid partners encode proteins that interact in the presence but not the absence of the third plasmid partner comprising at least one post-translational modifying enzyme. The expression of said enzyme is optionally under the control of an inducible transcriptional system. The first and second plasmid partners are selected by their ability to, in concert and in the presence of the expressed post-translational modifying enzyme, activate transcription of one or more marker genes in a host cell. In another embodiment, the first and second plasmid partners encode proteins that interact in the absence but not in the presence of the third plasmid partner.

Plasmids or vectors that encode all or a portion of a polypeptide of interest and polypeptides that interact with the polypeptide of interest are selected by contacting the array under conditions where the probe detectably hybridizes to complementary sequence, if present, within any of plasmids of the array. Plasmid partner or partners of the hybridized plasmid are identified (e.g., localized on the array) and optionally isolated, wherein said partner or partners encode a polypeptide capable of interacting, under physiological conditions and in a post-translational modification dependent manner, with the polypeptide of interest.

The post-translational modifiers selected for use in the third dimension of the 3PTH system can be selected from known genes encoding desired members of the family. For example, genes encoding several protein kinases have been cloned, such as PKA, SYK, p34cdc2, PKC and PI3 kinase and can be readily used in the method of the present invention by one of ordinary skill in the art. Similarly, genes for phosphatases, glycosylation enzymes and endoproteases have been cloned and can be readily used in the method of the present invention.

In another embodiment of the present invention, the modification enzyme encoded by the third plasmid is under the control of an inducible promoter such as MET25. Inducible promoters are well known in the art and can readily be incorporated into the method of the present invention. Other inducible systems include heat shock, GAL and tetracycline inducible promoter systems.

In one embodiment of the present invention, one or more post-translational enzymes are used in the third dimension of the 3PTH system. In a yet another embodiment of the present invention, the genes encoding post-translational modifiers of the third dimension can be a library of genes encoding such proteins. A family of kinases, proteases, glycosylation enzymes or endoproteases representing all or a portion of the cellular complement of such proteins can be generated, for example using PCR. For example, primers for PCR can be used that recognize known motifs in the class of post-translational modifier to be used in order to amplify all or a portion of the sequences encoding said modifiers. In one embodiment of the 3PTH method of the present invention, the third plasmid partner is not included in the arrays produced. In one embodiment, the first and second plasmid partners are selected using selective markers that is not present on the third plasmid partner.

In another embodiment, of the present invention, at least one plasmid partner is generated from a normalized library. Libraries can be normalized, for example, as described by Sive and St. John (Nucleic Acids Res. 16:10937 (1988)) and in U.S. Ser. No. 60/067,992 the teachings of which are both incorporated herein by reference in their entirety.

The plasmid partners can be generated by fusing the library sequences to DNA encoding one half of the selection complex e.g., the DNA binding domain sequence or transcription activation sequence such that a fusion protein comprising both segments is expressed. In one embodiment, the fusion is in frame between the coding sequence of both segments. However, libraries containing some out of frame fusions can be used, in which case the library is generally made with more clones. In one embodiment of the present invention, the library of the first plasmid partner is fused at its 5′ end to the sequence encoding a DNA binding protein. In another embodiment of the present invention, the library of the first plasmid partner is fused at its 3′ end to the sequence encoding a DNA binding protein. Similarly, the library of the second plasmid partner can be fused at either its 5′ or 3′ end to the sequence encoding the other half of the selection complex; for example, if the first plasmid partner uses the DNA binding domain, then the second plasmid partner uses the DNA transcription domain to generate the second plasmid partner.

In one embodiment of the present invention, the library comprises cDNA, full-length cDNA, genomic DNA or DNA encoding a peptide library. These libraries are readily available from commercial sources or can be synthesized using techniques well known in the art. For example, full length libraries are produced using methods described in U.S. Ser. No. 09/062,452 the teachings of which are incorporated herein by reference in their entirety.

The DNA binding protein and transcriptional activators used to generate the selection complex can be from any known transcriptional activation protein that binds DNA wherein the DNA binding domain binds in a sequence specific manner. Useful transcriptional activators are well known in the art. Particularly useful are those proteins where in the DNA activation domain and the DNA binding domain are separable at the DNA sequence level. In a further embodiment of the present invention, the DNA binding protein domain is selected from the group consisting of GAL4, lexA, GCN4 and ADR1. In another embodiment of the present invention, the transcription activation domain is selected from the group consisting of GAL4, GCN4, ADR1 and herpes simplex VP16.

The DNA binding site is typically placed upstream of a gene encoding a selectable marker for the host organism such that protein-protein interaction between the polypeptides encoded by the first and second partner plasmid results in transcription of the gene encoding the selectable marker. Selectable markers are well known in the art and include, for example, genes that render the host prototrophic for a given nutrient and genes that encode enzymes that produce a color or fluorescent product when exposed to the appropriate substrate.

In one embodiment, yeast clones harboring plasmid partners that encode interacting polypeptides are selected by growing the fused two-hybrid host on medium lacking the nutrient required in the absence of transcription of the gene encoding the selectable marker. In another embodiment the fused two-hybrid hosts are grown on medium containing the appropriate calorimetric or fluorogenic substrate for the enzyme encoded by the selecatable marker gene.

The two-hybrid hosts can be fused in batch. In one embodiment, the fused two-hybrid hosts are plated on the selective medium such that indvidual colonies are derived from individual fused hosts. Positive clones can be picked or isolated by hand or by robotic methods. Colony picking robots are well known in the art. In another embodiment, the fused two-hybrid hosts are contacted with the fluorgenic substrate and positive cells are selected using a fluorecence activated cell sorter. Methods of fluorescence activated cell sorting are well known in the art.

Suitable hosts for the two-hybrid screen of the present invention include any transformable organism that can be grown as a single-celled organism. Such hosts include prokaryotes and eukaryotes. In particular eukaryotic hosts can be yeast and mammalian or other cell culture.

The arrays of the present invention can be generated using polynucleic acid libraries from any organism of interest, including prokaryotic, archebacterial and eukaryotic organisms.

The present invention is drawn to a composition comprising an array of plasmids comprising two or more plasmid partners wherein a first plasmid partner comprises a first library fused to a nucleic acid encoding a DNA binding domain, a second plasmid partner comprises the first or a second library fused to a nucleic acid sequence encoding a transcriptional activation domain, wherein the first and second plasmid partners are selected to be in the array by their ability to, in concert and in the absence of expression of said post-translational modifying enzyme, activate transcription of one or more marker genes in a host cell, wherein the post-translational modifying enzyme is encoded by a third plasmid partner in the host cell.

The present invention is drawn to a composition comprising an array of plasmids comprising two or more plasmid partners wherein a first plasmid partner comprises a first library fused to a nucleic acid encoding a DNA binding domain, a second plasmid partner comprises the first or a second library fused to a nucleic acid sequence encoding a transcriptional activation domain, wherein the first and second plasmid partners are selected to be in the array by their ability to, in concert and in the presence of expression of said post-translational modifying enzyme, activate transcription of one or more marker genes in a host cell, wherein the post-translational modifying enzyme is encoded by a third plasmid partner in the host cell.

Methods for reducing the false negatives include making different libraries. For example, random primed cDNA two-hybrid libraries can be constructed to obtain small protein domains which may be buried in the intact proteins in a specific condition. Second, the protein fusion interface can be changed. Traditionally, the DNA binding domain and activation domain are located in the N-terminus of the fusion protein. New libraries can be constructed with the DNA binding domain and activation domain located at its C-terminus, so that the N-terminus of the bait protein can be free for its interactions. Third, the libraries for the first and second plasmid partner can be enriched for full-length genes. Furthermore, several “cytoplasm two-hybrid systems” have been developed. Cytoplasm two-hybrid systems can be integrated into the 3PTH system to cover those proteins which do not interact properly in the nucleus.

The invention will be further illustrated by the following non-limiting example.

### EXAMPLES

### Example 1

Three-protein Two Hybrid Screen

Plasmid Vectors:

Two vectors with three drug resistance genes are constructed. Each vector carries an unique E. coli selection marker such as Zeocin or DHFR (DHFR represents DiHydroFolate Reductase and confers resistant to Trimethoprin). The vectors also carry an additional common selection marker, β-lactamase. The drug resistant gene specific to each vector increases the efficiency of recovering the plasmids that are positive. The cycloheximide counterselection system (Harper et al., Cell, 75:805-816 (1993)) can be used to optimize selection.

The DNA-binding domain (DB) vector (or first plasmid partner) is constructed by inserting the Zeocin resistant gene into pGBT9 (Bartel et al., Methods Enzymol., 254:241-263 (1995)) or pDBTrp (Vidal, Bartel and Fields, Eds. Oxford Univ. Press 109, (1997)). pACT2 constitutes the basis of the activation domain (AD) vector (second plasmid partner) with the addition of the DHFR gene. pGBT9 and pDBTrp are selected because they yield low levels of false positives. While not wishing to be bound by theory, this may be due to their low level of gene expression. The main source of false positives usually originates from the activation of the DB-vector reporter gene by itself pDBTrp is a centromere-based (low copy number) expression plasmid with the full length ADH1 promoter. pGBT9 is a two micron-based (high copy number) expression vector with a truncation to give a minimal activity ADH1 promoter.

Yeast Strains:

The promoter strength of the reporter gene and the expression level of the two hybrid proteins determine the sensitivity of two-hybrid system. Thus, the selection of the yeast host strain is critical for success. In general, the upstream activating sequence (UAS) of GAL1 is stronger than GAL2 UAS, and GAL2 UAS is stronger than the synthetic GAL4 binding site consensus sequence (UAS G17-mer). The available host strains shown below are compared and the optimal pair is selected.

PJ69-2A: MATa, trp1-901, leu2-3,112, ura3-52, his3-200, gal4, gal80, LYS2::GAL1_{UAS}-GAL1_{TATA}-HIS3, GAL2_{UAS}-GAL2_{TATA}-ADE2 (James et al., Genetics 144:1425-1436 (1996))

Y187: MATα, ura3-52, his3-200,ade2-101, trp1-901, leu2-3,112, met-, gal4, gal80, URA3:: GAL1_{UAS}-GAL1_{TATA}-1acZ (Harper et al., Cell, 75:805-816 (1993)).

MaV103: MATa, leu2-3,112, trp1-901, his3_{—}200, ade2-101, gal4, gal80, SPAL10::URA3, GAL1(GAL1_{UAS})::1acZ, HIS3(GAL1_{UAS})::HIS3 @LYS2 (Vidal et al., Proc. Natl. Acad. Sci. USA 93:10315-10328 (1996)).

MaV203: MATα, leu2-3,112, trp1-901, his3_{—}200, ade2-101, gal4, gal80, SPAL10::URA3, GAL1(GAL1_{UAS})::lacZ, HIS3(GAL1_{UAS})::HIS3 @LYS2 (Vidal, 1997 )

The host strain pair PJ69-2A/Y 187 uses two different promoters (GAL 1 and GAL2) on three different reporters (HIS3, ADE2, lacZ). The yeast pair MaV103 and MaV203 has SPAL10(UAS G17-mer) and GAL1 as the promoters in front of reporters URA3, HIS3, and lacZ. The sensitivity of SPAL10 (UAS G17-mer) promoter to GAL 1 and GAL2 is compared by using a group of known interacting proteins with different affinities (Table I), then selecting the strain with stronger promoters.

TABLE I
Examples of Known Interacting Pairs

| | Hybrid #1 | Hybrid #2 | Interaction Strength |
|---|---|---|---|
| Pair 1 | human RB | human E2F | weak |
| | (aa302-928) | (aa342-437) | (Vidal et al., Proc. Natl. Acad Sci |
| | | | USA. 93(19).10315-20 (1996) |
| Pair 2 | Drosophila DP | Drosophila | moderate |
| | (aa1-377) | (aa225-433) | (Du et al., Genes Dev. |
| | | | 10(10):1206-18 (1996) |
| Pair 3 | cFos (aa132-211) | cJun (aa250-325) | strong |
| | | | (Chevray & Nathans, Proc. Natl. |
| | | | Acad. Sci., USA., 89 (13):5789-93 |
| | | | (1992)) |
| Pair 4 | murine p53 | 5V40 T antigen | moderate |
| | (aa72-390) | (aa87-708) | (Iwabuchi et al., Oncogene, |
| | | | 8(6):1693-6 (1993)) |
| Pair 5 | yeast SNF1 | yeast SNF4 | weak |
| | | | (Fields & Song, Nature 340: 245-6 |
| | | | (1989)) |
| Pair 6 | murine SNK | human CIB | moderate |
| | | | (Yuan & Erikson, unpublished) |

Library Construction:

A series of brain cDNA libraries is constructed using AlphaGene's normalization and FLEX™ (Full-Length Expressed gene) cDNA library construction technologies U.S. Ser. No. 09/062,452, the teachings of which are incorporated herein in their entirety. Plasmid pGBT9-Zeo has been constructed and tested by constructing a human fetal brain library with a titer of 1.8×10⁶ primary clones. The libraries were normalized. Table II summarizes the results of α-tubulin and β-actin abundance comparisons in two cDNA libraries, constructed from the same fetal brain mRNA. The protocol for library normalization is an improvement of the Sive and St. John protocol (Sive & St. john, 1988;). A short hybridization, corresponding to an estimated Cot of four, was carried out before addition of streptavidin to remove double-stranded DNA. Hybrid selection with alpha tubulin and beta actin was performed, followed standard procedures. In each hybrid selection an average of two thousand colonies were examined.

TABLE II
Library Normalization Data

| | Normalized Library | Control Library | Improvement |
|---|---|---|---|
| α-tubulin (%) | 0.1 | 0.9 | 9X |
| β-actin (%) | 0.1 | 0.7 | 7X |

Pre-screening:

The number of false positive clones is reduced by performing a prescreen. URA counterselection is performed to remove the false positive signal from the DNA-binding domain alone. The DNA-binding domain library are transformed separately into strain MaV203 and ura⁻ colonies are selected on 5-FOA plates. All surviving colonies are used for the two-hybrid interaction screening procedure.

Interaction Screen:

Yeast mating (Bendixen et al., Nucleic Acids Res. 22(9):1778-9. 1994) and plasmid transformation followed by nutritional selection are used in the two-hybrid interaction screen. The DNA-Binding domain (DB) library is transformed into strain PJ69-2 (a mating type a strain) and the Activation Domain (AD) library transformed into strain Y187 (a mating type α στραιν). The α and a transformants are mated with subsequent nutritional selection. Plasmids that grow after nutritional selection are isolated. Optimization of the mating/transformation step is critical because yeast cells, unlike E. coli, can acquire multiple plasmids following transformation. The amount of DNA used in transformation is varied to alter the number of plasmids transformed into cells.

For an initial test, pilot scale experiments are performed using two different approaches. Tests with 10 known interacting pairs with various affinities are conducted. The interactions among these 10 pairs are studied by 1) A matrix mating—the interaction of every possible pair (100 pairs in combination is examined); 2) A “library vs. library” or batch screen—the 10 clone pairs are mixed as two “mini-libraries” (one DB library and one AD library) followed by an interaction screen. The percentage of false positives and false negatives is determined for each selection marker.

Following the initial tests, a small scale genome wide interaction screen is performed with a pair of two-hybrid FLEX™ cDNA libraries. The ADE selection marker was chosen. To tighten the screen an additional marker, the E. coli lacZ gene can be used. There are two possible alternatives to select for the clones. In one scenario, the clones surviving nutritional selection are robotically picked to 96 well plates, followed by a liquid β-galactosidase assay with a chemiluminescent substrate (Campbell et al., 1995). In the second scenario, a β-galactosidase filter assay is performed and the blue colonies are robotically distributed in 96 well plates.

Plasmid Retrieval

Isolation of plasmids from yeast is not trivial. The problem is particularly difficult when working with large plasmids (>6 kb). Low yields and genomic contamination are common. To rapidly isolate the plasmid, 1.5 ml of saturated yeast cells were spun down and lysed in 10 μl of Lyticase for 60 min at 37° C., then 10 μl of 20% SDS was added with vigorous vortexing to help the cell lysis. The cells were put through one freeze/thaw cycle to ensure complete lysis. The whole cell lysate was passed through a spin column. The column beads of the high throughput spin column were purchased from Pharmacia Biotech (Sephacryl S-1000). The eluate from the spin column containing the purified DNA was collected for the transformation into E. coli for amplification. After the plasmid is isolated from yeast, the DNA is transformed back into E. coli for amplification. A multi-head electroporator from BTX, Genetronics, is used to increase the throughput. The two vector/three selectable markers system provides for efficient plasmid recovery.

Confirmation

Multiple E. coli transformants are picked, plasmid DNAs are isolated, and transformed back into yeast strains to confirm the interactions. Confirmation is necessary since yeast can carry multiple plasmids. It is worth noting that optimization of the plasmid transformation procedure may significantly lower the likelihood that a yeast cell carries more than one type of plasmid.

Construction of Arrays and Identification of Interacting Clones by Hybridization Amplification of the DNAs

A library versus library two-hybrid screen was performed as described above. The DNA-binding domain library was prescreened to remove clones that can activate the reporters in the absence of protein-protein interaction. Clones that passed the prescreen were mated with clones from an activation domain library. A portion of the mated cells were selected for those carrying protein-protein interactions. Plasmids from a portion of the selected colonies were retrieved from yeast cells and amplified in E. coli then extracted using standard molecular biology protocol.

One pair of interaction plasmids plus 10 known plasmids were spotted onto microarray slides in a duplicate fashion. Two independent clones from the 10 known genes were labeled with fluorescent CY3 or Cy5 for use as probes to determine whether the spotted plasmids can be correctly identified on microarray slides. Approximately 1 μg of each plasmid DNA was resuspended in 5× SSC buffer for printing (spotting) onto the slides. The printing procedure was followed according to the manufacturer's instructions. Approximately 6ng of plasmid DNA was printed onto a single spot in duplicate.

TABLE VI

| Locations | Plasmid Clone | | |
|---|---|---|---|
| (duplicate) | Name | Insert Description | Comment |
| A1, B1 | YY367-1 | B11 in pGBT9 | B11 is a novel gene |
| | | vector | previously identified |
| | | | as interacting with |
| | | | Snk (at A4 and B4) |
| A2, B2 | F2 | F2 in pGBT9 | Interacts with F14 at |
| | | | A5 and B5 |
| A3, B3 | Alpha 4 | Alpha 4 in | Interacts with PP6 at |
| | | pGBT9 | A6 and B6 |
| A4, B4 | YY89-1 | Snk in an | Snk, a protein kinase, |
| | | activation domain | interacts with B11 at |
| | | vector | A1 and B1 |
| | | (pGAD424) | |
| A5, B5 | F14 | F14 in pGAD | Interacts with F2 at |
| | | vector | A2 and B2 |
| A6, B6 | PP6 | PP6 in pGAD | Interacts with Alpha 1 |
| | | vector | At A3 and B3 |
| A7, B7 | YY313-9 | B11 in pGAD | B11 is a novel gene |
| | | vector | previously identified |
| | | | as interacting with |
| | | | Snk (at A4 and B4) |
| A8, B8 | TD-1 | SV40 large T- | |
| | | antigen in pACT2 | |
| A9, B9 | B75 | B75 in pGAD | |
| | | vector | |
| A10, B10 | A18 | A18 in pGAD | |
| | | vector | |
| C1, C3 | 2-hybrid clone 1 | An unknown | interacts with clone2 |
| | | clone | at C2 and C4 |
| | | in DAN-binding | |
| | | domain vector | |
| C2, C4 | 2-hybrid clone2 | An unknown | interacts with clone1 |
| | | clone in | at C1 and C3 |
| | | activation | |
| | | domain vector | |

Fluorescent Probe Synthesis

Genes B11 and Snk were excised from clones YY313-9 and YY8-11 respectively and the inserts were isolated from agarose gel by centrifugation. Approximate 25ng of denatured DNAs were labeled by Cy3 and Cy5 dCTP (purchased from Amersham Biotech) in a reaction containing random primer mixture and reaction buffer (both provided by High Prime DNA Labeling Kit from Boehringer Mannheim), 25 μM 2′-deoxyadenosine-5′-triphosphate, 25 μM 2′thymidine-5′-triphosphate, 25 μM 2′-deoxyguanosine-5′-triphosphate, 5 μM 2′-deoxycytidine-5′-triphosphate and 20 μM Cy3 or Cy5 labeled 2′-deoxycytidine-5′-triphosphate and 4 U of Klenow polymerase. The reaction was incubated at 37° C. for 45 min and stopped by incubating at 65° C. for 10 min. The probes were purified by standard ethanol precipitation and resuspended in 10 μl of hybridization buffer (6× SSC, 5× Denhart's solution, 2% SDS, 0.1 μg/μl of yeast tRNA).

Hybridization

2 μl each of Cy3 probe and Cy5 probe were combined for hybridizing the DNA on each of glass slide. The slides were placed in slide chambers with a towel wet with 2× SSC. They were brought up to 80° C. for 10 min and then immediately put on ice to denature the DNA, hybridized at 62° C. for 6 hours, then washed to remove the unhybridized probes by 2× SSC, dried, and scanned by GenePix 4000 Microarray Scanner from Axon Instruments.

Results

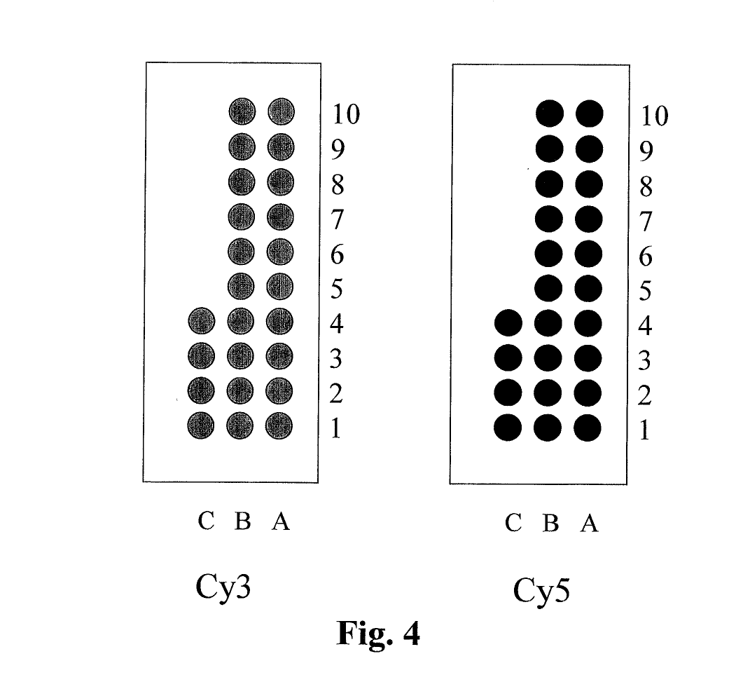

1. To evaluate the spotting procedure, a Cy5 or Cy3 labeled probe containing common vector sequences was hybridized to the DNA on glass slide. FIG. 4 showed that all DNAs were attached onto slide.

2. To localize the clone Snk

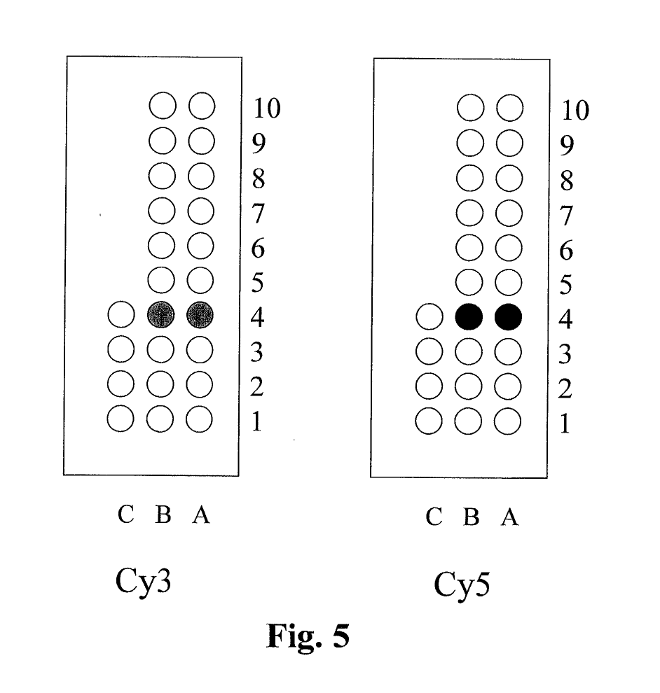

A Snk probe either labeled by Cy3 or Cy5 was hybridized to the slide. FIG. 5 shows Snk probe hybridizes both A4 and B4 DNAs which are the Snk clones.

3. To localize the clone B 11:

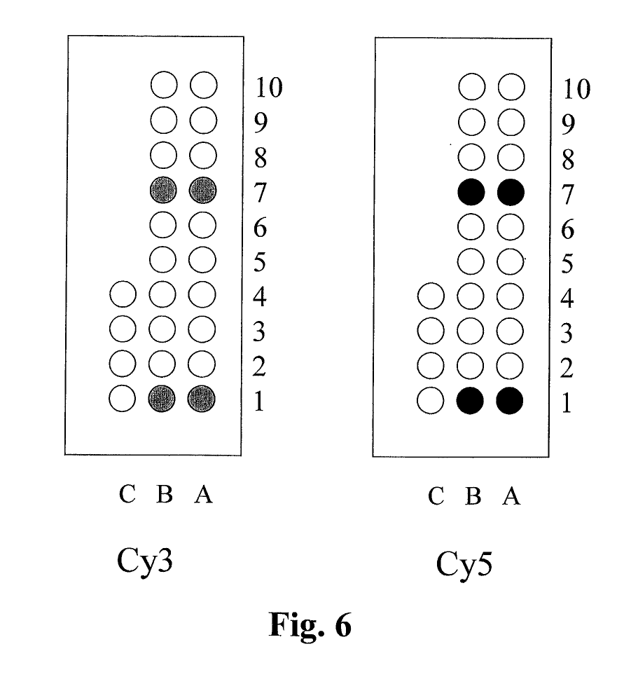

A B 11 probe either labeled by Cy3 or Cy5 was hybridized to the slide. FIG. 6 shows the B11 probe hybridizes locations A1, B1, A7, B7 (location 1 represents B11 in pGBT9 and location 7 represents B11 in the pGAD vector).

Thus, a specific DNA probe can correctly identify homologous DNA on an array with little or no background. For example, if Snk is the polypeptide of interest, a labeled Snk polynucleotide is used to probe the interaction array. The hybridization identifies A4 and B4. From the linkage information shown in Table VI, the clone at A1 and B 1 (B 11) is identified as a Snk interacting clone. The linkage information is provided by the arrays of the present invention. This method can be used repetitively with newly identified interacting clones as the probes, to draw a protein interaction map and establish biological interaction pathways.

### Example 2

Construction of Two Hybrid Arrays

Plasmid vectors:

A new vector is constructed to be used as the third plasmid partner. This vector carries URA3 marker, a yeast 2μ origin of replication, a yeast nuclear localization signal, multiple cloning sites, ampicillin section marker and Col E1 origin of replication. A yeast inducible promoter (Met25). A constitutive promoter (pADH) can also be used.

Yeast Strains:

A new ∝ mating type strain is constructed from Y187 the URA3 phenotype is reverted to ura3 by 5-Fluoroorotic Acid (5-FOA) counterselection. The desired geontype is (Mat∝, ura3, his3, ade2, trp1, leu2, met, gal4Δ, gal80Δ, ura3::GAL1ᵤₐₛ- GAL1_{TATA}-lacZ). PJ69-2A (a mating type A strain) will be used in the mating assay as the second plasmid partner.

Library construction:

A series of brain cDNA libraries is constructed using technology described U.S. Pat. Nos. 5,162,290, 5,643,766 and Ser. No.: 09/062,452, the teachings of which are incorporated herein in their entirety. The bait library is cloned into the pGBT9 derived vector and the prey library is cloned into the PACT2-derived vector. Kinases are chosen for the third plasmid partner in 3PTH. Kinases are chosen to obtain sufficient activity. Kinases are selected based on the following criteria, either separately applied or applied in combination: (1) kinases that have been overexpressed in the host cell in the past; (2) kinases whose constitutive and/or inactive forms are available; (3) kinases which have homologous pathways in yeast (allowing activation in yeast by an endogenous activator if necessary); (4) kinases which are expressed in a tissue of interest (for example, brain tissue). The nucleic acid encoding the kinase or kinases are expressed under the control of an inducible promoter such as the MET25 inducible promoter.

The presence or absence of kinase activity under specified conditions is optimized using positive control pairs of proteins as shown in Table III. The first column represents the first hybrid protein, the second column represents the second hybrid protein, the third column represents the third plasmid partner (kinase) and the fourth column represents the affinity of interaction after the hybrid protein is phosphorylated by the kinase listed in column 3.

TABLE III

| | | | Interaction |
|---|---|---|---|
| Hybrid 1 | Hybrid 2 | Kinase | Level¹ |
| CREB | CBP | PKA | increase |
| IgE receptor | SH2-B | Syk or Lyn | increase |
| RGSZ1 | Gzalpha | PKC | increase |
| HsEg5 | dynactin (p150) | P34cdc2 | increase |
| NMDA receptor | calmodulin | PKC | decrease |
| Mu2 | CTLA-4 | P13 | decrease |

¹of phosphorylated form

3PTH Library Screening:

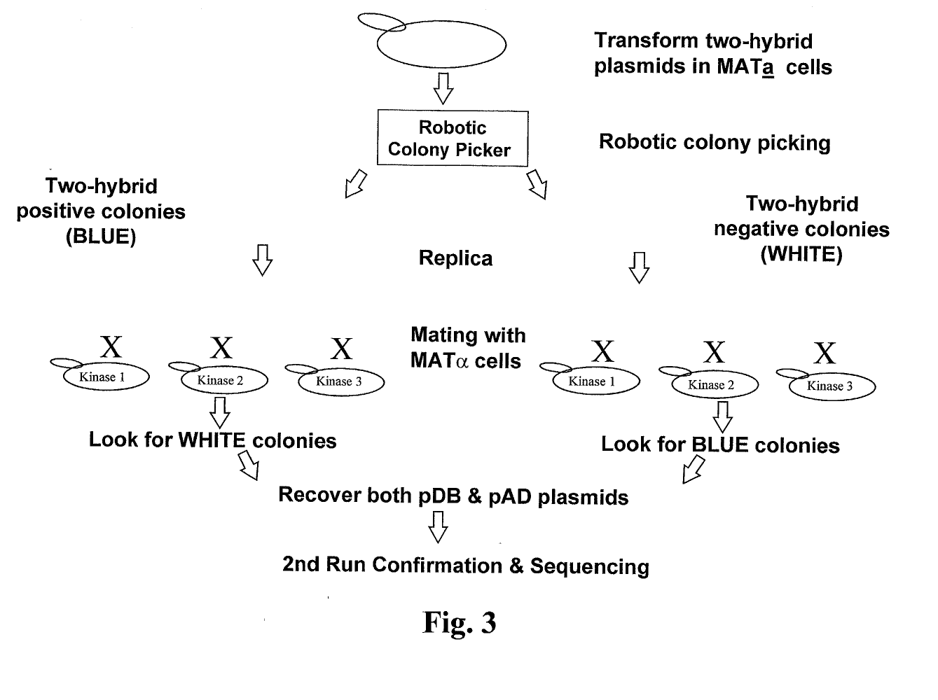

Both bait and prey fusion protein containing plasmids are transformed into one haploid yeast strain (MATa, for example) and the kinase containing plasimid(s) are transformed into the other haploid strain (MATα). FIG. 3 is a flow chart of the 3PTH system.

Screen for Interactions that Occur Only in the Unphosphorylated Form:

Both the pGBT9 based library and the PACT2 based library DNA are transformed into ura3 MATa cells having the ade⁻ genotype. White (ADE⁺) colonies are picked and arrayed onto 96 well plates by a robot. A control panel, including positive and negative controls are also placed onto each plate. The arrays of cells are grown and replica plated onto the desired number of plates (e.g. the number of kinases to be screened plus one plate for a negative control of empty kinase vector). The kinase vectors (carrying the URA3 marker) are transformed into MAT∝ cells.

The MATa cells and MAT∝ cells, transformed as described above, are mated in batch fashion. White colonies are selected and the GBT9 and pGAD424 fusion plasmids are recovered by Zeocin and Ampicillin selection, respectively, using standard plasmid isolation techniques. The desired clones have the following phenotypes:

TABLE IV

| Plasmid | Color in 3PTH Assay |
|---|---|
| GBT9 alone | red |
| GAD424 alone | red |
| GBT9 + GAD424 | white |
| GBT9 + GAD424 + kinase | red |

DNA encoding proteins that interact with a polypeptide of interest only in the absence of phosphorylation are selected and isolated by probing the array of plasmids recovered above with DNA encoding the polypetide of interest.

Screen for Interactions that Occur Only in the Presence of Phosphorylation

The initial screen for interacting proteins is performed as describe above except red transformed MATa colonies are picked by the robot. The kinase transformed MAT∝ cells are mated to the selected, transformed MATa cells in batch fashion. The HIS+ and ADE+ colonies are arrayed onto 96 well plates. The arrays are replicated onto two plates. On one plate, kinase expression is turned off by adding methanol to turn of the MET2 promoter, or by adding 5-FOA to counter select the URA plasmid. The cells on the other plate are allowed to express the kinase. Colonies that are white on the kinase⁺ plate and red on the kinase⁻ plate are selected. Plasmids are recovered as described above. The desired clones have the following phenotypes:

TABLE V

| Plasmid | Color in 3PTH Assay |
|---|---|
| GBT9 alone | red |
| GAD424 alone | red |
| GBT9 + GAD424 | red |
| Kinase + GBT9 | red |
| Kinase + GAD424 | red |
| GBT + GAD424 + kinase | white |

DNA encoding proteins that interact with a polypeptide of interest only in the presence of phosphorylation are selected and isolated by probing the array of plasmids selected above with DNA encoding the polypetide of interest.

Equivalents

Those skilled in the art will recognize, or be able to ascertain using no more than routine experimentation, many equivalents to the specific embodiments of the invention described herein. Such equivalents are intended to be encompassed by the following claims.

what is claimed is:

1. A method of identifying polynucleic acid encoding at least one polypeptide capable of interacting, in vivo with a polypeptide of interest comprising;
a) contacting at least one array of plasmids with a nucleic acid probe, wherein;
i) said probe encodes the polypeptide of interest or fragment thereof, wherein said probe hybridizes to complementary sequence, if present, within any of the plasmids of the array, and wherein;
ii) said array comprises two or more plasmid partners, wherein a first plasmid partner comprises a first library fused to a first nucleic acid sequence encoding a first half of a selection pair and a second plasmid partner comprises the same or a second library fused to a second nucleic acid sequence encoding a second half of a selection pair wherein the plasmid partners are selected to be in the array by their ability to produce active selection pair in a host cell and wherein the plasmid partners are present in the array at known locations, and;
b) detecting probe hybridized to the array, thereby identifying polynucleic acid encoding at least one polypeptide capable of interacting, in vivo, with the polypeptide of interest.

2. The method of claim 1, wherein the selection pair comprises a DNA binding domain and a transcriptional activation domain.

3. The method of claim 2, wherein the DNA binding domain sequence is selected from the group consisting of: GAL, lexA, GCN4 and ADR1.

4. The method of claim 2, wherein the transcription activation domain sequence is selected from the group consisting of: GAL, GCN4, ADR1 and herpes simplex VP16.

5. The method of claim 1, wherein the first library is normalized.

6. The method of claim 1, wherein the second library is normalized.

7. The method of claim 1, wherein the library of the first plasmid partner is fused at its 5′ end to the first nucleic acid sequence.

8. The method of claim 1, wherein the library of the first plasmid partner is fused at its 3′ end to the first nucleic acid sequence.

9. The method of claim 1, wherein the library of the second plasmid partner is fused at its 3′ end to the second nucleic acid sequence.

10. The method of claim 1, wherein the library of the second plasmid partner is fused at its 5′ end to the second nucleic acid sequence.

11. The method of claim 1, wherein the plasmid partners are in separate linked arrays.

12. The method of claim 1, wherein the plasmid partners are together in the same array.

13. The method of claim 1, wherein the host cell is a prokaryotic cell.

14. The method of claim 1, wherein the host cell is an eukaryotic cell.

15. The method of claim 1, wherein a) further comprises a third plasmid comprising a sequence encoding at least one post-translational modifying enzyme.

16. The method of claim 15, wherein the post-translational modifying enzyme is under the control of an inducible promoter system.

17. The method of claim 15, wherein the post-translational modifying enzyme is selected from the group consisting of kinases, phosphatases, glycosylation enzymes and endoproteases.

18. The array of claim 1, wherein the array comprises at least one set of plasmids comprising two or more plasmid partners, wherein a first plasmid partner comprises a first library fused a first nucleic acid sequence encoding a first half of a selection pair and a second plasmid partner comprising the same or a second library fused to a second nucleic acid sequence encoding a second half of a selection pair, and wherein the plasmid partners are selected to be in the array by their ability to, in concert, activate the selection pair in a host cell.

19. A method of identifying polynucleic acid encoding at least one polypeptide capable of interacting, in vivo, with a polypeptide of interest, wherein the interaction is affected by post-translational modification, comprising;
a) contacting at least one array with a nucleic acid probe, wherein;
i) said probe encodes the polypeptide of interest or fragment thereof, wherein said probe hybridizes to complementary sequence, if present, within any of the plasmids of the array, and wherein;
ii) said array comprises two or more plasmid partners, wherein a first plasmid partner comprises a first library fused to a first nucleic acid sequence encoding a first half of a selection pair, a second plasmid partner comprising the first or a second library fused to a nucleic acid sequence encoding a second half of a selection pair and a third plasmid partner comprising a polynucleic acid sequence encoding at least one post-translational modifying enzyme, wherein the first and second plasmid partners are selected by their ability to produce active selection pair in a host cell in a manner dependent upon the third plasmid partner and wherein the first and second plasmid partners are present in the array at known locations; and
b) detecting probe hybridized to the array, thereby identifying polynucleic acid encoding at least one polypeptide capable of interacting, in vivo, with polypeptide of interest.

20. The method of claim 19, wherein production of active selection pair occurs in the presence of post-translation modify enzyme encoded by the third plasmid partner.

21. The method of claim 20, wherein the selection pair comprises a DNA binding domain and a transcriptional activation domain.

22. The method of claim 21, wherein the DNA binding protein domain sequence is selected from the group consisting of: GAL, 1exA, GCN4 and ADR1.

23. The method of claim 21, wherein the transcription activation domain sequence is selected from the group consisting of: GAL, GCN4, ADR1 and herpes simplex VP16.

24. The method of claim 19, wherein the first library is normalized.

25. The method of claim 19, wherein the second library is normalized.

26. The method of claim 19, wherein the library of the first plasmid partner is fused at its 5′ end to the first nucleic acid sequence.

27. The method of claim 19, wherein the library of the first plasmid partner is fused at its 3′ end to the first nucleic acid sequence.

28. The method of claim 19, wherein the library of the second plasmid partner is fused at its 3′ end to the second nucleic acid sequence.

29. The method of claim 19, wherein the library of the second plasmid partner is fused at its 5′ end to the second nucleic acid sequence.

30. The method of claim 19, wherein the plasmid partners are in separate linked arrays.

31. The method of claim 19, wherein the plasmid partners are together in the same array.

32. The method of claim 19, wherein the post-translational modifying enzyme is selected from the group consisting of: kinases, phosphatases, glycosylation enzymes and endoproteases.

33. The method of claim 19, wherein the post-translational modifying enzyme is under the control of an inducible promoter system.

34. The method of claim 33, wherein the inducible transcriptional system is selected from the list consisting of: MET25, heat shock, GAL and tetracycline sensitive promoters.

35. The method of claim 19, wherein the interaction is inhibited by the post-translational modification.

36. The array of claim 19, wherein the array comprises at least one set of plasmids comprising two or more plasmid partners, wherein a first plasmid partner comprises a first library fused to a first nucleic acid sequence encoding a first half of a selection pair and a second plasmid partner comprising the same or a second library fused to a second nucleic acid sequence encoding a second half of a selection pair, and wherein the plasmid partners are selected to be in the array by their ability to, in concert, activate the selection pair in a host cell.

37. A composition comprising at least one array of plasmids comprising two or more plasmid partners at known locations, wherein a first plasmid partner comprises a first library fused to a first nucleic acid sequence encoding a first half of a selection pair and a second plasmid partner comprising the same or a second library fused to a second nucleic acid sequence encoding a second half of a selection pair, and wherein the plasmid partners are selected to be in the array by their ability to, in concert, activate the selection pair in a host cell.

38. A composition comprising an array of plasmids comprising two or more plasmid partners at known locations wherein a first plasmid partner comprises a first library fused to a nucleic acid encoding a DNA binding domain, a second plasmid partner comprises the first or a second library fused to a nucleic acid sequence encoding a transcriptional activation domain, wherein the first and second plasmid partners are selected to be in the array by their ability to, in concert and in the absence of expression of post-translational modifying enzyme, activate transcription of one or more marker genes in a host cell, wherein said post-translational modifying enzyme is encoded by a third plasmid partner in the host cell.

39. A composition comprising an array of plasmids comprising two or more plasmid partners at known locations wherein a first plasmid partner comprises a first library fused to a nucleic acid encoding a DNA binding domain, a second plasmid partner comprises the first or a second library fused to a nucleic acid sequence encoding a transcriptional activation domain, wherein the first and second plasmid partners are selected to be in the array by their ability to, in concert and in the presence of expression of post-translational modifying enzyme, activate transcription of one or more marker genes in a host cell, wherein said post-translational modifying enzyme is encoded by a third plasmid partner in the host cell.

40. The composition of claim 39, wherein the selection pair comprises a DNA binding domain and a transcriptional activation domain.

41. The composition of claim 40, wherein the DNA binding protein domain sequence is selected from the group consisting of: GAL, lexA, GCN4 and ADR1.

42. The composition of claim 39, wherein the first library is normalized.

43. The composition of claim 39, wherein the second library is normalized.

44. The composition of claim 39, wherein the library of the first plasmid partner is fused at its 5′ end to the first nucleic acid sequence.

45. The composition of claim 39, wherein the transcription activation domain sequence is selected from the group consisting of: GAL, GCN4, ADR1 and herpes simplex VP16.

46. The composition of claim 39, wherein the library of the first plasmid partner is fused at its 3′ end to the first nucleic acid sequence.

47. The composition of claim 39, wherein the library of the second plasmid partner is fused at its 3′ end to the second nucleic acid sequence.

48. The composition of claim 39, wherein the library of the second plasmid partner is fused at its 5′ end to the second nucleic sequence.

49. The composition of claim 39, wherein the plasmid partners are in separate linked arrays.

50. The composition of claim 39, wherein the plasmid partners are together in the same array.

51. The composition of claim 39, wherein the post-translational modifying enzyme is selected from the group consisting of kinases, phosphatases, glycosylation enzymes and endoproteases.

52. The composition of claim 39, wherein the post-translational modifying enzyme is under the control of an inducible promoter.

53. The composition of claim 52, wherein the inducible promoter is selected from the list consisting of: MET25, heat shock, GAL and tetracycline sensitive promoters.

54. A kit comprising at least one array of plasmids comprising two or more plasmid partners at known locations, wherein a first plasmid partner comprises a first library fused to a first nucleic acid sequence encoding a first half of a selection pair and a second plasmid partner comprising the same or a second library fused to a second nucleic acid sequence encoding a second half of a selection pair, and wherein the plasmid partners are selected to be in the array by their ability to, in concert, activate the selection pair in a host cell and buffers for hybridizing polynucleic acids of interest to said array, and instructions for hybridization.

55. A method of generating an array of plasmids comprising;
a) conducting a two-hybrid screen, wherein a bait plasmid comprises a cDNA library and a prey plasmid comprises the same or a second cDNA library;
b) selecting positive two-hybrid clones and;
c) immobilizing the bait and prey plasmids from said positive clones on a solid support at known locations, thereby generating an array of plasmids.
